(19) United States
(12) Patent Application Publication
(10) Pub. No.: US 2022/0113736 A1
(43) Pub. Date: Apr. 14, 2022

(54) A METHOD OF TREATING A ROOM USING A ROBOTIC, MOBILE APPARATUS

(71) Applicant: Gama Healthcare Ltd., Watford (GB)

(72) Inventors: Guy Braverman, Watford (GB); Philip Samuel Johnson, Watford (GB); Filipe Horta, Watford (GB); James William Williamson, Leicester (GB)

(73) Assignee: Gama Healthcare Ltd., Watford (GB)

(21) Appl. No.: 17/424,241

(22) PCT Filed: Jan. 21, 2020
(86) PCT No.: PCT/EP2020/025026

§ 371 Date: Jul. 20, 2021

(30) Foreign Application Priority Data
Jan. 22, 2019 (GB) 1900859.8

(51) Int. Cl. G05D 1/02; A61L 2/10; A61L 2/20; A61L 2/24; G05D 1/00

(52) U.S. Cl. CPC G05D 1/0238; A61L 2/10; A61L 2/208; A61L 2/24; G05D 2201/0206; G05D 1/0274; A61L 2202/16; A61L 2202/25; G05D 1/0044

(57) ABSTRACT

A method of treating an enclosed space, in particular a room in a hospital, is provided, for example by disinfecting same, using a robotic mobile apparatus emitting ultraviolet (UV-C) radiation or hydrogen peroxide vapour (HPV). The robotic, mobile apparatus (1) comprises a wheeled carriage (3) on which a treatment device (2) is mounted and a controller (14) is provided that is configured to control operation of the wheels (12) of the carriage (3) and to operate the treatment device (2). A human-machine interface (HMI) (5) is also provided that is in communication with the controller (14) and is operable to start and stop a treatment procedure. A route around the enclosed space is recorded in the computer memory from a start position to an end position. When a treatment procedure is started, the human-machine interface (5) initiates operation of the controller (14), which controls operation of the wheels (12) of the carriage (3) so that the wheeled carriage (3) robotically tracks along the route around the enclosed space (53) and which controls operation of the treatment device (2) during its track along said route. The treatment may comprise disinfection by using a plurality of UV-C emitting lamps (10) mounted on the wheeled carriage.

The present invention relates to a method of treating an enclosed space, in particular a room in a hospital, for example by disinfecting same using a robotic mobile apparatus emitting ultraviolet (UV-C) radiation or hydrogen peroxide vapour (HPV).

Infectious microbe strains that are resistant to antibiotics and chemical disinfectants are a growing threat to the general public. Hospitals and clinics are particularly prone to harbouring these dangerous microbes, which pose a considerable danger to patients that have weakened immune systems. To counter these microbes in a manner which prevents their acquiring resistance, the use of apparatus which irradiates them with high frequency ultraviolet radiation (UV-C) is becoming more common. This is because electric bulbs that produce UV-C radiation with wavelengths in the range 2800 Å to 150 Å are now widely available. Such bulbs have been incorporated into hospital building structures in order that they can be operated remotely in empty rooms to sterilize the room. They have also been incorporated into transportable, free-standing apparatus for placement into rooms requiring disinfection.

Hydrogen peroxide vapour (HPV) fogging is also a new and growing method used to disinfect hospital rooms.

It will be appreciated that both methods of disinfection require the apparatus employed to be used remotely in an enclosed space, such as a closed room or closed-off portion of a hospital corridor, so that they do not pose a danger to personnel. As hospital rooms have a complexity compounded by their need to contain beds, trolleys, curtains and medical equipment, it is not always possible to provide effective disinfection from a single location within the room. In view of this it is important to ensure that the disinfection apparatus operates effectively and disinfects all parts of the space in which it operates.

It is an object of the present invention to provide a method of using a mobile treatment apparatus that will operate robotically and move around an enclosed space while in operation with view to providing an efficient treatment that treats all parts of the enclosed space without operator intervention being required.

It should be appreciated that while such treatment is described herein as disinfection, this is only as an example as the robotic apparatus of the invention may be used to provide other forms of treatment.

According to the present invention there is provided a method of treating an enclosed space using a robotic, mobile apparatus comprising

providing a wheeled carriage with omni-directional, controllable wheels that are adapted to be individually motor driven;

providing a treatment device mounted on the wheeled carriage;

providing a programmable controller with a computer memory configured to control operation of the wheels of the carriage and to operate the treatment device;

providing a human-machine interface (HMI) in communication with the controller and operable to start and stop a treatment procedure;

the method comprising

locating the wheeled carriage and treatment device within the enclosed space;

positioning the human-machine interface (HMI) outside the enclosed space;

recording a route around the enclosed space in the computer memory from a start position to an end position;

initiating operation of the controller via the human-machine interface (HMI) to commence operation of the treatment device and to operate the wheels of the carriage so that the wheeled carriage robotically tracks along the route around the enclosed space.

Preferably, the route around the enclosed space is recorded in the computer memory device by an operator moving the wheeled carriage around the enclosed space from the start position to the end position, the position, direction of movement and speed of the wheels of the carriage being recorded in the computer memory by the controller during said movement of the wheeled carriage along the route; and after initiation of the controller via the human-machine interface (HMI) to commence operation of the treatment device the controller operates the wheeled carriage to robotically track back along the route from the end position back to the start position by controlling the position, direction of movement and speed of the wheels of the carriage based on the recorded position, direction of movement and speed of the wheels of the carriage during recordal of the route.

Alternatively, a light detection and ranging surveying device is provided and mounted on the wheeled carriage, the method comprising the additional steps of creating a three-dimensional map of the enclosed space by operation of the light detection and ranging surveying device, viewing the map on a display device of the human-machine interface (HMI) and creating the route, and initiating operation of the controller via the human-machine interface (HMI) to commence operation of the treatment device the operate the wheeled carriage to track robotically along the route by comparing the position of the wheeled carriage as detected by the light detection and ranging surveying device with a desired position along the route and by controlling the position, direction of movement and speed of the wheels of the carriage.

Other preferred but non-essential features of the present invention are described in the dependent claims appended hereto.

The present invention will now be described by way of example with reference to the accompanying drawings, in which:

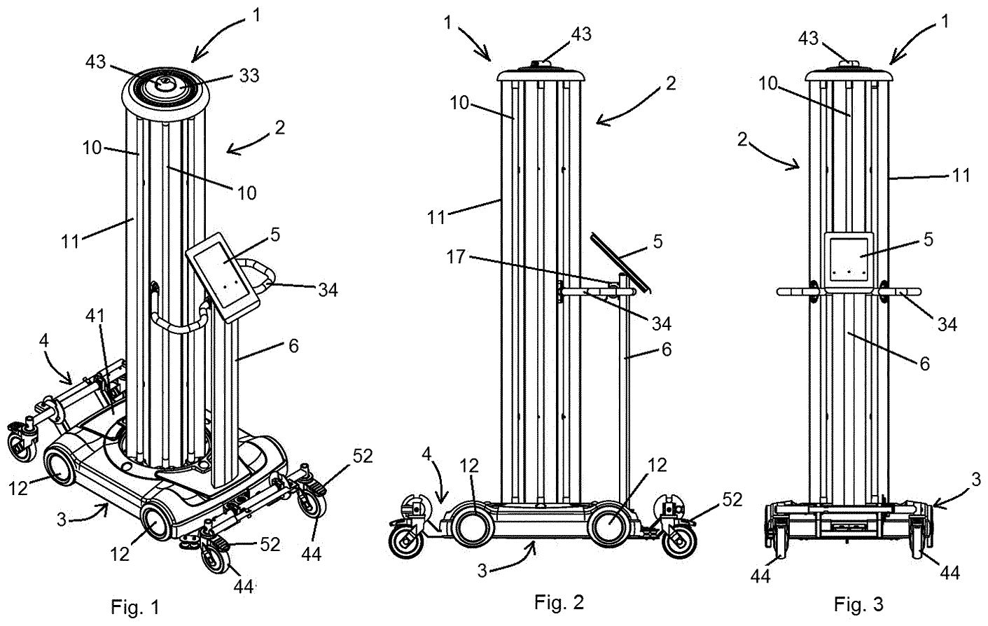

FIG. 1 is a perspective view of an embodiment of a robotic, mobile apparatus in accordance with the present invention;

FIG. 2 is a side view of the apparatus shown in FIG. 1;

FIG. 3 is a rear view of the apparatus shown in FIGS. 1 and 2;

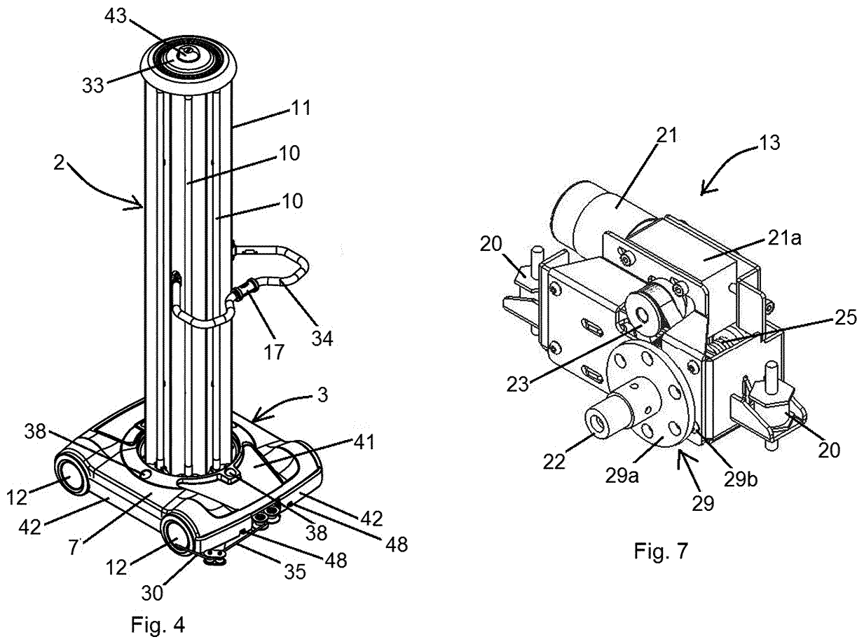

FIG. 4 is a perspective view of a disinfection apparatus forming part of the apparatus shown in FIGS. 1 to 3;

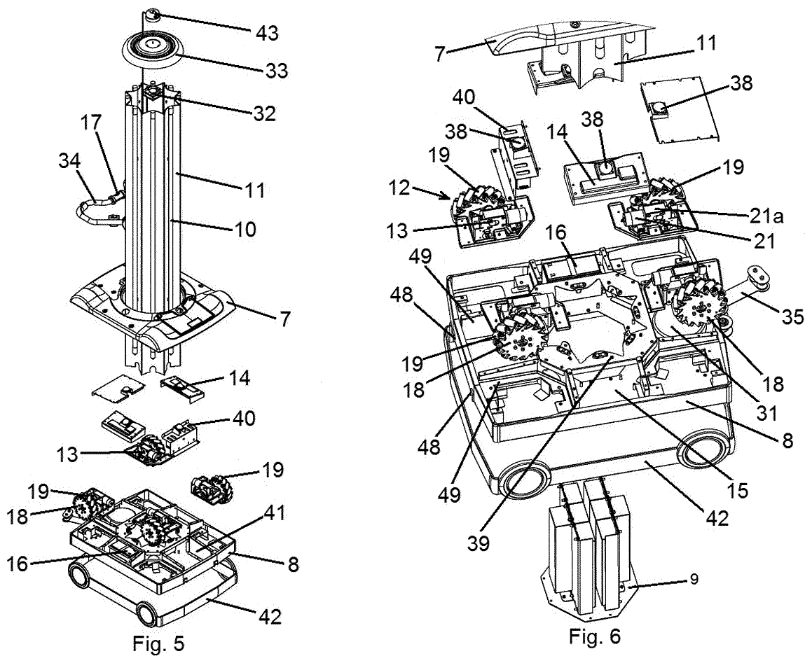

FIG. 5 is an exploded perspective view of the apparatus shown in FIG. 4 but without any UV-C lamps;

FIG. 6 is an exploded view to an enlarged scale of a wheeled carriage forming part of the apparatus shown in FIG. 5;

FIG. 7 is a perspective view of a drive unit for a wheel of the wheeled carriage;

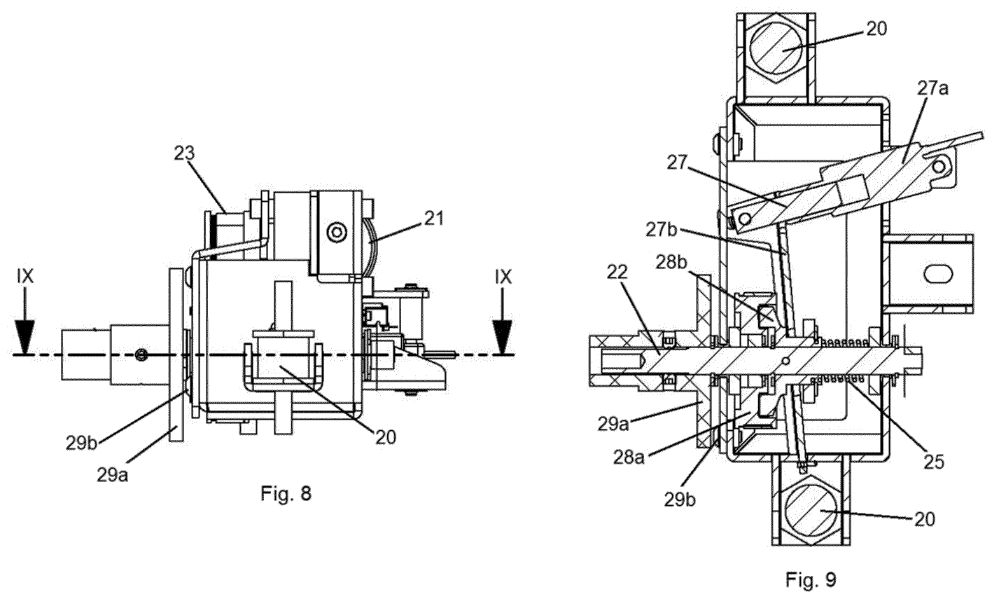

FIG. 8 is an end view of the drive unit shown in FIG. 7;

FIG. 9 is a cross-sectional view along the line IX-IX in FIG. 8;

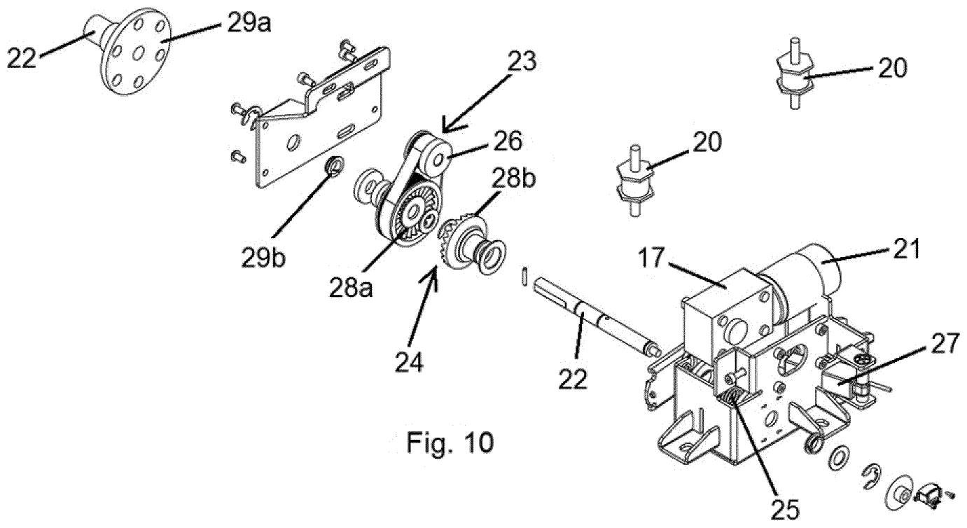

FIG. 10 is an exploded view of the drive unit;

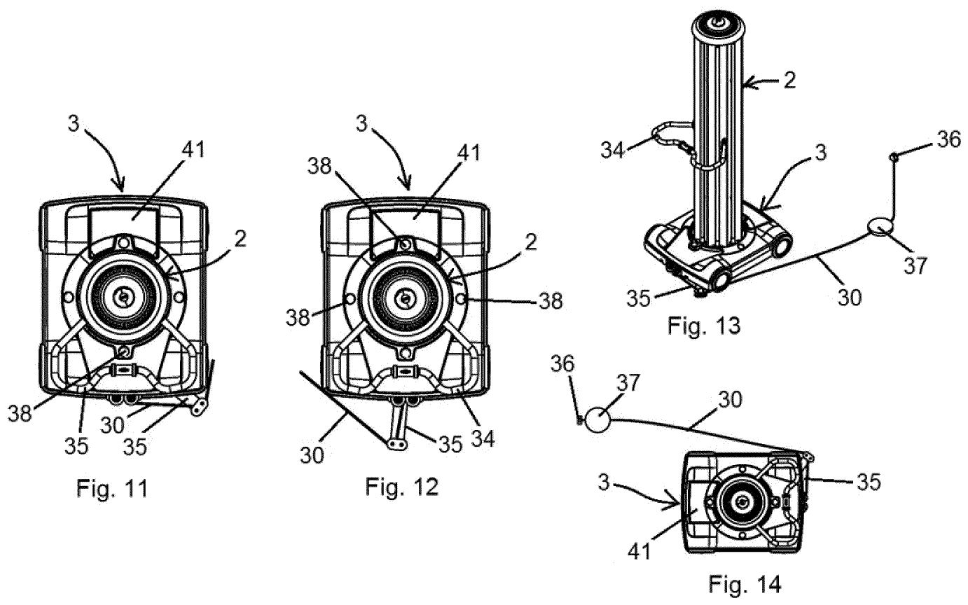

FIG. 11 is a plan view of the disinfection apparatus shown in FIGS. 1 to 4 with an arm of a cable management system shown in one position;

FIG. 12 is a view similar to FIG. 11 but with the arm of the cable management system shown in another position;

FIG. 13 is a perspective view of the disinfection apparatus with an electrical cable for same shown in a position when plugged into a wall socket;

FIG. 14 is a plan view of FIG. 13;

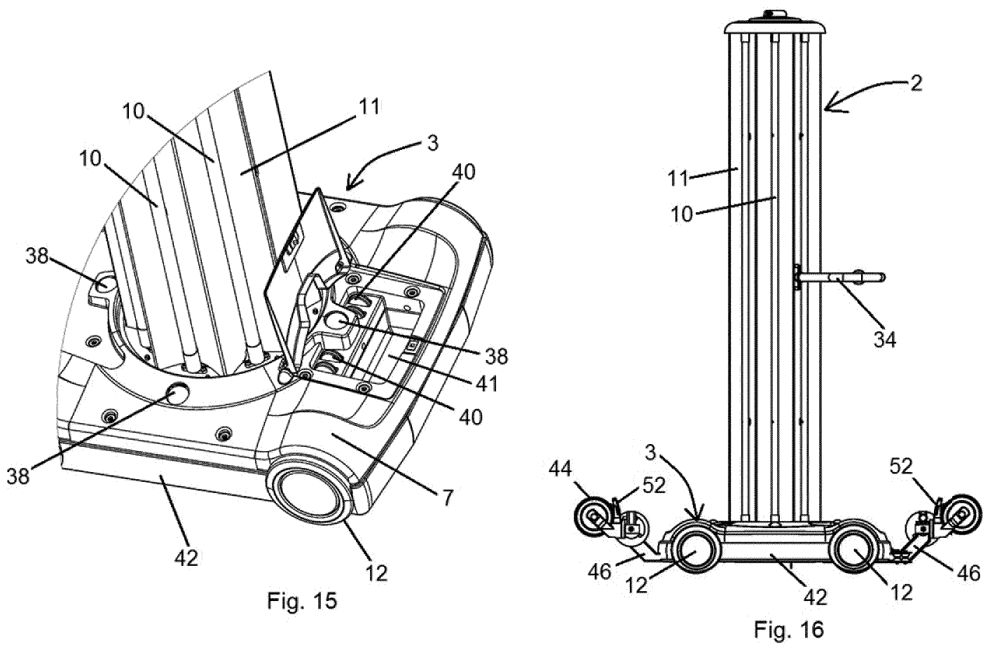

FIG. 15 is a view to an enlarged scale of part of the wheeled carriage with a lid of a compartment thereof shown open;

FIG. 16 is a side view of the disinfection apparatus when connected to a transportation trolley;

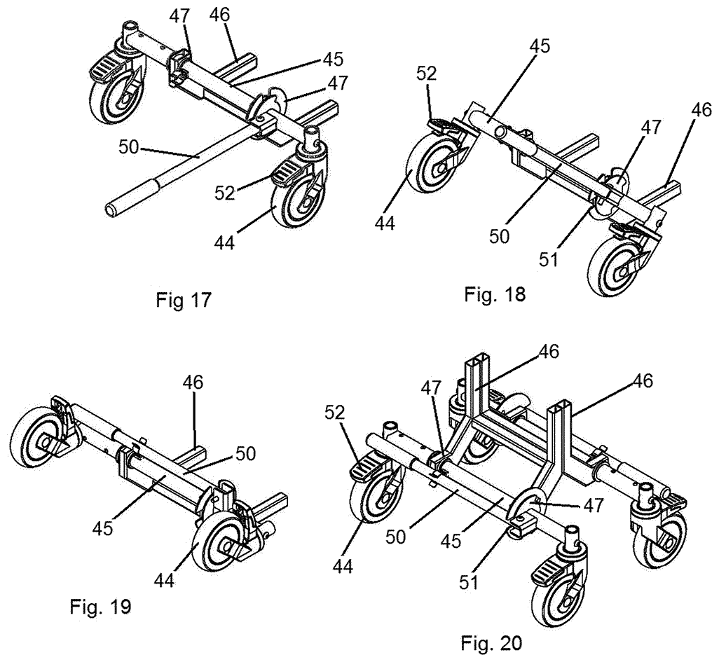

FIG. 17 is a perspective view of one half of the transportation trolley shown in a position wherein its wheel in a position in contact with a floor surface;

FIG. 18 is a view similar to FIG. 18 but showing the trolley with its wheels partially raised above the floor surface;

FIG. 19 is a view similar to FIG. 18 but with the wheels fully raised and locked in position;

FIG. 20 is a perspective view of two halves of the transportation trolley when connected together;

FIGS. 21 to 26 are schematic views showing a sequence of events during operation of the apparatus in accordance with the invention when treating an enclosed space containing a bed;

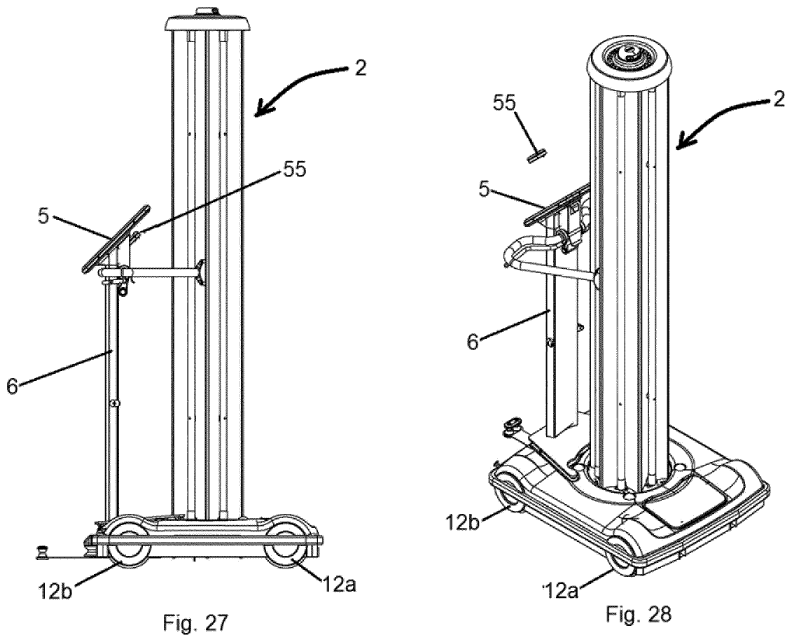

FIG. 27 is a side view of a robotic, mobile apparatus similar to that shown in FIGS. 1 to 3 but when modified by the addition of a detachable timer; and

FIG. 28 is perspective view of the apparatus shown in FIG. 27 when the timer has been detached from the apparatus.

The illustrated embodiments of the invention are of methods and appropriate apparatus for use in the methods that treating an enclosed space by disinfection, in particular by UV-C irradiation. However, as indicated above, the method of the invention may be used to provide other forms treatment and the following description should be read in this light, in particular where the terms “disinfecting” and “disinfection device” are used.

A robotic, mobile apparatus 1 for disinfecting an enclosed space is shown in FIGS. 1 to 3. The apparatus 1 comprises an assembly of a disinfection device 2 that is mounted on a wheeled carriage 3, which together is hereinafter termed “the disinfection apparatus” and is shown so/us in FIG. 4, a transportation trolley 4, on which the wheeled carriage 3 may be removably mounted and from which it is demounted prior to use of the disinfection device 2, and a human-machine interface (HMI) 5 for controlling operation of the apparatus 1. The human-machine interface 5 is provided on a free-standing unit 6 that may be docked on the wheeled carriage 3 for transportation as shown in FIGS. 1 to 3.

The disinfection device 2 in the illustrated embodiment is shown as a device that uses ultraviolet (UV-C) radiation for the disinfection of an enclosed space but other forms of disinfection device could be used instead, for example a device that uses hydrogen peroxide vapour (HPV) fogging or other forms of radiation. These devices are conventional and their particular method of operation need not be described herein.

With reference to the illustrated embodiment the apparatus 1 is designed to disinfect an enclosed space, such as a hospital room by irradiating the space with UV-C radiation. To this end and as is described in more detail below, in use the unit 6 comprising the human-machine interface 5 is undocked from the carriage 3 and located outside the space or room to be irradiated. The rest of the apparatus is then wheeled into the space or room and the transportation trolley 4 detached from the wheeled carriage 3, which is then left standing on the floor of the space or room. It is necessary to plot a predetermined route around the space or room that the disinfection apparatus will follow during operation of the disinfection device 2. This is to ensure that all parts of the room will be effectively disinfected during the treatment procedure. The plotting is carried out during an encoding phase by manoeuvring the disinfection apparatus around the desired route so that its movements can be encoded and recorded or by programming the route into the apparatus 1 beforehand. Once the space or room has been sealed, the disinfection device 2 is be switched on and the wheeled carriage 3 set in motion so that it robotically tracks along the predetermined route around the enclosed space or room to disinfect same.

The various parts of the apparatus 1 and its method of operation will now be described in more detail with particular reference to FIGS. 5 and 6.

The wheeled carriage 3 is self-propelled and comprises a casing 7 covering a substantially rectangular framework 8 to which the various components of the apparatus 1 housed within the carriage 2 are attached. Mounted on a plate 9 secured at the centre of the framework 8 is the disinfection device 2, which in the present embodiment is a UV-C disinfecting device that comprises a plurality of tubular UV-C emitting lamps 10 that are vertically mounted around a central column 11. The column 11 is secured to the plate 9 and extends above the carriage 2. Preferably, the external surface of the column 11 is shiny and each lamp 10 is located in its own concave portion of the column 11, which portion provides a reflector for that lamp 11. Respectively mounted at the four corners of the framework 8 are four omni-directional, controllable wheels 12. The wheels 12 are preferably mecanum wheels that are each provided with their own drive unit 13 that is linked to a controller 14 housed within an enclosure 15 of the framework 8.

The controller 14 is programmable and under the wireless control of the HMI 5, which may itself be independently programmable. The controller 14 and the HMI 5 may also be adapted to communicate with a remote monitoring station that is set up to monitor several apparatuses 1, for example all those used within a specific building, such as a hospital. In this way use of the apparatus 1 can be monitored and validated as described below.

The wheels 12 and therefore the carriage 3 is powered by a rechargeable main battery 16 via signals from the controller 14 whereby each wheel 12 can be powered independently of the others. The wheeled carriage 2 is therefore self-propelled and operates robotically, as is described below. The main battery 16 is also used to charge one or more rechargeable daughter batteries in the HMI 5 when the latter is docked on the carriage 3 via a socket 17 into which one or more connectors on the HMI 5 may be plugged.

The mecanum wheels 12 are conventional and each comprises an inner wheel 18 around the circumference of which are attached a series of rollers 19 each having an axis of rotation at 45° to the plane of the wheel 18 and at 45° to a line through its centre parallel to the axis of rotation of the wheel 18. By alternating wheels 12 with left and right-handed rollers 19 in such a way that each wheel 12 applies force roughly at right angles to the wheelbase diagonal to which the wheel 12 is mounted, the carriage 3 can be made to move in any direction and turn by varying the speed and direction of rotation of each wheel 12. Moving all four wheels 12 in the same direction causes forward or backward movement, running the wheels 12 on one side of the carriage 3 in the opposite direction to those on the other side causes rotation of the carriage 3, and running the wheels 12 on one diagonal in the opposite direction to those on the other diagonal causes sideways movement of the carriage 3. Hence, combinations of these wheel motions allow for motion of the carriage 3 in any direction in addition to any desired carriage rotation.

The drive units 13 that control operation of each of the wheels 12 are identical in structure and shown in detail in FIGS. 7 to 10. They are mounted via soft damping mounts 20 to the framework 8 and each comprise a motor 21 that is powered by the battery 16. The motor 21 drives a shaft 22 via a gearbox 21a. The shaft 22 has an associated stub axle to which the wheel 12 is connected, via a belt drive 23. A toothed clutch mechanism 24 is provided that is biased via a spring-loading 25 against a driven pulley 26 of the belt drive 23. In an alternative arrangement, the belt drive 23 may be replaced by a gear drive (not shown) that comprises a gear wheel against which the toothed clutch mechanism 24 is biased. The clutch mechanism 24 may be disengaged by an actuator 27 which acts against the bias of the spring-loading 25 to disengage intermeshing toothed wheels 28a and 28b of the clutch mechanism 24. The actuator 27 may comprise a linear actuator or a solenoid 27a that on operation shortens the length of the actuator 27 and pulls back a bracket 27b to which the toothed wheel 28b is connected, thereby disconnecting it from the toothed wheel 28a and disengaging the clutch mechanism 24. Operation of the actuator 27 is under the control of the controller 14. When it is operated and the clutch mechanism 24 is disengaged free movement of the wheel 12 is permitted. The clutch mechanism 24 is also linked to an encoder 29 comprising a disc 29a and associated sensor 29b that together are used to sense the movement of the wheel 12 over time during an encoding phase of operation of the drive unit 13 when free movement of the wheels 12 of the carriage 3 is required. This is in order that the wheels 12 can be driven in a reverse motion by the motor 21 during a playback phase of operation of the drive unit 13 when the clutch mechanism 24 is engaged, as is described in more detail below. Data relating to the movement of the wheels 12 recorded by the encoder disc 29 is transmitted to and stored by the controller 14 during the encoding phase for recall during the playback phase of operation.

Turning now to the UV-C disinfecting device 2, this comprises eight UV-C emitting, tubular lamps 10 that are mounted in the concave reflectors formed by the central column 11, which is hollow. More or fewer lamps 10 may be provided in other embodiments of the device 2. The lamps 10 are adapted to be powered by a mains electrical supply via a cable 30, which is stored on a retractable cable reel 31 housed in the carriage 3. The column 11 is hollow in order that a cooling airflow can be created through the column 11 when the lamps 10 are operational by means of a fan 32 that is mounted at the top of the column 11 beneath a perforated plate 33 that closes off the top of the column 11. Although in the present embodiment the fan 32 draws air into and down through the column 11, in other embodiments a fan or the fan 32 could blow air upwards through the column 11. The clean air drawn into the column 11 is expelled through holes is the column 11 to cool the lamps 10. In addition, the column 11 itself, which will typically be made of aluminium, acts as a large heat sink.

A projecting rail 34 forming two handgrips is secured to the column 11 to allow the wheeled carriage 3 to be manoeuvred over a floor surface and moved around a predetermined route during the encoding phase as is further described below.

As the wheeled carriage 3 is designed to operate robotically, it is important that during operation the cable 30 does not become entangled around the wheeled carriage 3 during operation of the apparatus 1. This is prevented by the provision of a cable management system that controls the tension of the cable 30. In particular, the cable 30 is tensioned, preferably by a constant force spring provided within the reel 31, and is threaded through the free end of an arm 35 that is pivotally mounted on and extends from one side of the carriage 3, typically the rear of the carriage. The arm 35 is freely rotatable about its pivot and is long enough to allow the cable 30 to be guided and kept clear of the wheels 12 when the carriage 3 moves, as shown in FIGS. 11 and 12 wherein the arm 35 is shown in two different positions respectively. As the arm 35 rotates its length is such that it guides the cable 30 around wheeled carriage 3 and keeps the cable 30 from being entangled by the wheels 12 even when the cable 30 is in front of the motion of the carriage 3.

As the cable 30 will be plugged into a mains electrical supply, it is also important to reduce strain on its plug 36 as the carriage 3 moves around robotically when in use. To this end a restraint is preferably provided that retains the electrical cable 30 against or close to a floor surface at a location close to the plug 36. The restraint may comprise a weight 37 that weighs the electrical cable 30 against the floor surface. The weight 37 may be provided with wedges and/or a clip (not shown) so that it can be secured to the cable 30. Alternatively, the restraint may comprise a clamp that secures the electrical cable 30 to a fixed location such as the wheel of a bed or other fitting within the enclosed space or room that is close to the floor and to the an electrical socket into which the plug 36 is plugged. As the apparatus 1 is most likely to be used in a hospital where electrical sockets are provided at positions that are a considerable distance from a floor surface, the restraint holds the cable 30 close to the floor to prevent it from being a trip hazard. In addition, retaining the cable 30 close to the floor ensures that in use it wraps around the wheels 12 and does get snagged in their operating mechanisms under the carriage 3.

When the disinfection device 2 is in operation within an enclosed space, it is important that the enclosed space, for example a room within a hospital, is evacuated of all persons and animals as the UV-C light emitted by the lamps 10 is a danger to health. The wheeled carriage is therefore preferably provided with at least one sensor 38, such as a passive, infrared-based motion sensor (a PIR sensor). The sensor 38 detects the movement of people, animals, and other objects and is linked to the controller 14, which acts to prevent operation of the carriage 3 and the lamps 10 if the sensor 38 detects the presence of persons or animals within the enclosed space. Preferably, a plurality of sensors 38 are provided and spaced around the base of the column 11 in the carriage 3 in order that no part of the enclosed space is ever hidden from any one of the sensor's fields of operation.

It is also important to ensure that the UV-C lamps 10 both operate and operate correctly by emitting the correct intensity of UV-C radiation, in particular because the lamps 10 are only operated when there is no one in the vicinity of the lamps 10 and they cannot be seen. To this end, a plurality of first UV sensors 39 may be mounted in fixed positions on the wheeled carriage 3 and linked to the controller 14. The sensors 39 are located beneath the casing and such that each only receives UV-C radiation from a respective one of the lamps 10 and is used to monitor the level of this radiation when the lamps 10 are operation. The information received by the sensors 39 is related to the controller 14 and thence to the HIV interface 5 and/or directly to a central monitoring station that collates the operation of a plurality of apparatus 1. Should any lamp 10 fail to operate or not operate correctly, this can be flagged up by the HIV interface 5 or by the central monitoring station in order that the malfunctioning lamp 10 can be replaced.

It is also important to ensure that all parts of an enclosed space receive the correct dosage of UV-C radiation to ensure that the space has been adequately disinfected after use of the apparatus 1. A plurality of autonomous, second UV sensors 40 may also be provided and stored in a lidded compartment 41 provided for them in the carriage 3. The sensors 40 are preferably battery operated and may each include a rechargeable battery that is charged from the main battery 16 when they are plugged respectively into a plurality of sockets provided for this purpose within the compartment 41, as shown in FIG. 15. The compartment 41 may also be used for the storage of other items, for example a cover for the treatment device 2 when not in use and the weight 37.

These sensors 40 may be used intermittently in order to validate the operation of the apparatus 1 by being placed at various locations in an enclosed space prior to disinfection of the space by operation of the apparatus 1. The sensors 40 are adapted to detect the level of UV-C radiation received and relay this information either to the HIV interface or to the central monitoring station. The operation of the disinfecting device may therefore be validated. In particular and as is detailed below, the route taken by the carriage 3 around an enclosed space during operation of the lamps 10 may be adjusted based on the validation information received and transmitted by the sensors 40.

In order to prevent the carriage 3 from continuing to try to move robotically should it encounter an obstacle in its path, a pressure sensitive cushioned tape switch 42 is located around the vertical side walls of the casing 7. This switch 42 is connected to the controller 14, which acts to stop operation of the wheels 12 and operation of the disinfection device 2 should an unexpected obstacle be encountered during use. In these circumstances the controller 14 also signals to the human-machine interface 5 that an obstacle has been encountered so that the problem can be sorted out.

It will be appreciated that the controller 14, the HMI 5 and powered components within the carriage 3 are battery operated, either directly from the main battery 16 or via daughter batteries recharged from the main battery 16. In contrast, the disinfection device 2 is powered from the mains. As the batteries are rechargeable, the mains supply to the disinfection device 2 is used to recharge the main battery 16 when the disinfection device 2 is operational. In turn, the main battery 16 is used to recharge the daughter batteries when the disinfection device is switched off, for example during transportation of the apparatus 1 or when it is in storage. The controller 14 is preferably programmed to ensure recharging of the daughter batteries is initiated as appropriate.

A light detection and ranging surveying device 43 may be mounted on the plate 33 at the top of the column 11. Such devices are usually termed “LiDar” units and the unit 33 is linked to the controller 14 and under the control of the HMI 5. It is operated in order that a three-dimensional map of an enclosed space to be disinfected by the apparatus 1 can be created. This enables a preferred route around the space to be predetermined and programmed into the controller 14 via the human-machine interface 5 so that the wheeled carriage 3 can be operated to follow same during operation of the disinfection device 2 without having to be tracked over the route in an encoding phase beforehand.

The transportation trolley 4 is provided so that the apparatus 1 can be transported when not in use between various locations within a building without the wheels 12 of the carriage 3 having to come into contact with contaminated floor surfaces during said transportation. The trolley 4 also reduces unnecessary wear on the wheels 12. The trolley 4 is made in two parts and each part comprises a pair of castor wheels 44 that are mounted at the end of a connecting rod 45. The rod 45 is connectable to the wheeled carriage 3 in order to raise one side of the wheeled carriage 3 above a floor surface so that the two parts of the trolley 4 are fitted on opposite sides, typically front and rear sides, of the wheeled carriage 3. The connection is made by a pair of cranked bars 46, which are rotatably mounted on the rods 45 by clamps 47 so that the bars 46 in each pair are spaced, parallel and have free ends that project at 90° from the rod 45 to which they are connected. These ends are adapted to be inserted into channels 48 provided in strengthening ribs 49 of the framework 8 of the carriage 3. Each rod 45 is also provided with a handle 50 that is pivoted thereto so that it can be folded away parallel with the rod 45 but that can be rotated so that it extends at 90° to the rod 45. When the handle 50 is folded away it engages in a slot 51 provided in one of the clamps 47 and thereby locks the rod 45 against rotation relative to the bars 46. Hence, when the free ends of the bars 46 have been inserted into the channels 48, the handles 50 can be pivoted outwards and used to rotate the rods 45 to lower the castor wheels 44 to raise the carriage 3 from the floor. The handles 50 can then be folded away thereby locking the wheels 44 in place in their lowered position. In this position the apparatus 1 is readily manoeuvrable using the rail 34 without risk of contamination to the wheels 12 of the carriage. Each of the castor wheels 44 is also provided with a brake pedal 52.

Prior to use of the disinfection device 2, the transportation trolley can be removed from the carriage 3 by firstly pivoting the handles 50 and rotating the rod 45 to raise the wheels 44 and to lower the carriage 3 so that the wheels 12 contact the floor surface. The bars 46 can then be removed from the channels 48 and the transportation trolley stowed away while the sterilization device 2 is in use. Preferably, the two parts of the transportation trolley 4 can be clipped together for easy transportation, as shown in FIG. 20.

Prior to the treatment of an enclosed space, such as a room 53 in a hospital containing a bed 54, it is necessary to determine a suitable route along which the carriage 3 should robotically track to provide an effective and efficient treatment. This route is then encoded in the controller 14 or transmitted to the controller 14, which can then operate to control operation of the wheels 12 of the carriage 3 so that the carriage 3 will move along the route.

A method of encoding this route and of a subsequent method of treatment will now be described with reference to FIGS. 21 to 26.

First, an apparatus 1 in accordance with the invention and comprising the assembly of the disinfection device 2 and carriage 3, the transportation trolley 4 and the human-machine interface (HMI) 5, as shown in FIGS. 1 to 3 is wheeled to a position outside the enclosed space, in this example the room 53, to be disinfected, as shown in FIG. 21. The free-standing unit 6 comprising the HMI 5 is then undocked from the carriage 3 and stood outside the room 53. The rest of the apparatus 1 is then wheeled into the room and parked at a location at one side of the room, preferably near an electrical socket, as shown in FIG. 22. The transportation trolley 4 is then removed from the carriage 3, which is lowered onto the floor. The trolley 4 is preferably taken outside the room 53 and the electrical cable 30 of the disinfection device 2 is plugged into a mains socket. The weight 37 is then preferably attached to the cable 30 in a position close to the socket so that the cable is anchored to the floor.

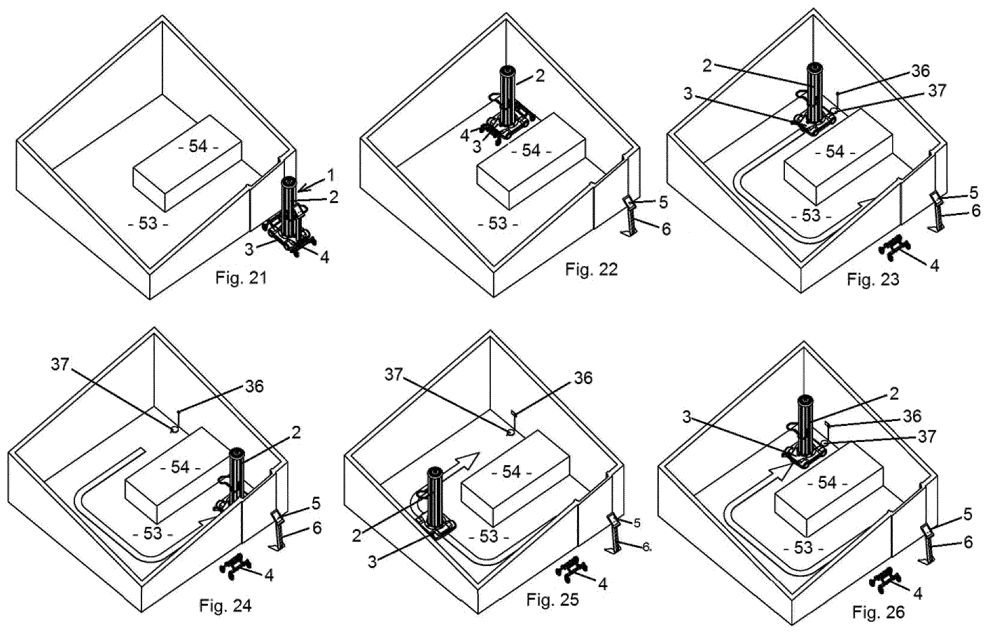

Using the HMI 5 or a switch on the carriage 3, the disinfection apparatus is placed into a record mode wherein the encoders 29 in the drive units 13 will record the movement of the wheels 12. The disinfection apparatus is then manually wheeled around the room 53 tracing a desired route, as shown by the arrow in FIG. 23, that will permit UV-C radiation emanating from the lamps 10 to reach all parts of the room 53 for an appropriate length of time to achieve disinfection when the lamps 10 are operational. It may be necessary for the operator to move pieces of movable furniture or other obstacles in the path of the disinfection apparatus around the room 53 to create an optimal route. It may also be appropriate for the operator to establish positions along the desired route where the device 2 is stationary and dwells for a predetermined time in order to ensure that all parts of the space or room receive appropriate irradiation by the lamps 10. Once the end of the optimal route is reached, the operator should evacuate the room 53 leaving the disinfection apparatus in place at the end of the route, as shown in FIG. 24. The door of the room 53 should be closed to enclose the room and then the disinfection apparatus can be switched into a playback mode wherein it becomes operational via the HMI 5.

In the playback mode, the disinfection device 2 is activated from the HMI 5 so that the lamps 10 are switched on and the wheels 12 of the carriage 3 are operated via the controller 14 so that they follow the movements recorded during the encoding phase but in reverse. The carriage 3 therefore robotically tracks back along the predetermined route from its end to its beginning, as shown by the arrow in FIG. 25. When the disinfection device 2 reaches the beginning of the route, as shown in FIG. 26, that is after robotically tracking back along it, the controller 14 alerts the HMI 5 and switches off the lamps 10 of the disinfection device. The controller 14 also operates the actuator 27 so that the wheels 12 can free-wheel. The room 53 can now be safely entered to retrieve the disinfection device, which is wheeled out of the room, the transportation trolley 4 attached to the carriage 3 and the HMI 5 docked back on the carriage 3.

During the disinfection process, the sensors 38 and 39 are in operation to ensure that the lamps 10 are switched off if any motion is detected within the room 53 and that the lamps 10 are operating correctly. From time to time, the operation of the apparatus 1 can be monitored for any given shape of room 53 by deploying the autonomous sensors 40.

It will be appreciated that in more sophisticated embodiments of the apparatus 1, the LiDar unit 43 can be used to provide a map of the room that is displayed on the HMI 5. An operator can then draw a preferred route for the disinfection apparatus to follow over the map and the controller 14 instructed to operate the wheels 12 of the carriage 3 to follow the predetermined route. The interchange of information between the controller 14 and the HMI 5 permits this to happen as the LiDar unit 43 is aware of the position of the disinfection unit within the room 53.

All of the information gleaned from the sensors 38, 39 and 40 and information relating to the predetermined route for any given shape of room can be stored for future use and for monitoring and validation purposes in a computer memory device within the controller 14, the free-standing unit 6 and/or at a remote monitoring station with which the apparatus 1 may communicate wirelessly.

Hence, the present invention provides a mobile treatment apparatus that will operate robotically and move around an enclosed space while in operation with view to providing an efficient treatment that treats all parts of the enclosed space without operator intervention being required during the treatment.

Turning now to FIGS. 27 and 28, the apparatus 1 may be modified by the provision of a detachable, portable timer 55 that docks into the human-machine interface (HMI) 5. The timer 55 is provided in order that it can be detached from the HMI 5 after the device 2 has been activated and provide an alarm, for example by buzzing, vibrating and/or flashing, when the treatment procedure has been carried out by the device 2. Hence, the timer 55 can be carried by an operator who can engage in other tasks during the treatment procedure and be alerted by the timer 55 when the treatment procedure is finished so that he or she can return to the apparatus 1 to deploy it elsewhere. Preferably, the timer 55 is also adapted to indicate at any given time the remaining runtime of the treatment procedure.

The timer 55 is battery powered, the battery preferably being rechargeable in which case the timer 55 plugs into the HMI 5 and is charged at the same time as the rechargeable daughter batteries in the HMI 5 by the main battery 16 when the HMI 5 is docked on the carriage 3. A projected time for the treatment procedure may be calculated by the controller and transmitted to the timer 55 by Wi-Fi. Alternatively, the timer 55 may be adapted to receive start and stop signals from the controller 14 by Wi-Fi on commencement and after completion of a treatment procedure respectively. In all cases the timer 55 is adapted to provide the alarm either after the projected time has elapsed or following receipt of a stop signal.

The projected time for the treatment procedure may be calculated by the controller 14 and/or HMI 5 in one of several ways. All of these use the path length travelled by the device 2, which is calculated using the known circumference of the wheels 12, the number of pulses from the encoders 29, which may be 500 per wheel rotation, and the trace speed, which is the speed of movement of the device 2 when in treatment mode and which is assumed to be constant. This data is cross-referenced with the required motor speed to calculate the time taken by the device to travel the predetermined route and then dwell times at the start and end of the treatment procedure are added to this to obtain the total treatment time. This time is then sent to the portable timer 55 by Wi-Fi once a treatment procedure is started. The dwell times cover the time required at the start of the treatment procedure for the lamps 10 to warm-up prior to the start of movement of the carriage 3 along the predetermined path and the time required at the end of the path when the lamps 10 remain operational after the carriage 3 has stopped moving to ensure that all parts of the room 53 receive adequate UV irradiation.

Five possible ways of calculating the time taken by the device 2 to travel the predetermined route are as follows.
1. The highest number of pulses of all four wheels 12 during the record mode is taken and are broken into irregular segments in which the speed of movement of the device 2 during record mode is substantially constant. Projected treatment times for each of these segments is calculated using the trace speed and an appropriate fraction of the speed recorded during record mode, it being appreciated that during record mode an operator is likely to move the device 2 faster than the motors 21 can drive the device 2 during treatment mode, These times are then added together to give the total time to travel the predetermined route.
2. The average number of pulses of the two front wheels 12a (see FIG. 27) of the device 2 is recorded and times calculated for a plurality of segments based on the trace speed and a fraction of the speed during record mode for each segment as indicated in 1. above. These time are then added together to give the total time to travel the predetermined route.
3. The average number of pulses of the two rear wheels 12b (see FIG. 27) of the device 2 is recorded and times calculated for a plurality of segments based on the trace speed and a fraction of the speed during record mode for each segment as indicated in 1 above. These times are then added together to give the total time to travel the predetermined route.
4. The average number of pulses of all four wheels 12 of the device 2 is taken and times calculated for a plurality of segments based on the trace speed and a fraction of the speed during record mode for each segment. These time are then added together to give the total time to travel the predetermined route.
5. The speed of the centre of the device 2 is recorded along with the angles of the wheels 12 at each of a plurality of intervals and integrated over time to give the length of the predetermined path. As the trace speed is known for each interval, the total time to travel the predetermined route can be calculated.

1-15. (canceled)

16. A method of treating an enclosed space using a robotic, mobile apparatus, the method comprising:
providing a wheeled carriage with omni-directional, controllable wheels that are adapted to be motor driven;
providing a treatment device mounted on the wheeled carriage;
providing a LIDAR (Light Detection and Ranging) unit configured to be aware of the position of the wheeled carriage;
providing a programmable controller with a computer memory configured to control operation of the wheels of the carriage and to operate the treatment device;
providing a human-machine interface (HMI) in communication with the controller and operable to start and stop a treatment procedure;
the method comprising
locating the wheeled carriage and treatment device within the enclosed space;
positioning the human-machine interface (HMI) outside the enclosed space;
recording a route around the enclosed space in the computer memory from a start position to an end position;
initiating operation of the controller via the human-machine interface (HMI) to commence operation of the treatment device and to operate the wheels of the carriage so that the wheeled carriage robotically tracks along the route around the enclosed space, and further using the LIDAR unit to confirm that the wheeled carriage is tracking along the route around the enclosed space as intended.

17. A method as claimed in claim 16, wherein
the route around the enclosed space is recorded in the computer memory device by an operator moving the wheeled carriage around the enclosed space from the start position to the end position, the position, direction of movement and speed of the wheels of the carriage being recorded in the computer memory by the controller during said movement of the wheeled carriage along the route; and
after initiation of the controller via the human-machine interface (HMI) to commence operation of the treatment device the controller operates the wheeled carriage to robotically track back along the route from the end position back to the start position by controlling the position, direction of movement and speed of the wheels of the carriage based on the recorded position, direction of movement and speed of the wheels of the carriage during recordal of the route.

18. A method as claimed in claim 16, wherein
a light detection and ranging surveying device is provided and mounted on the wheeled carriage, the method comprising the additional steps of creating a three-dimensional map of the enclosed space by operation of the light detection and ranging surveying device, viewing the map on a display device of the human-machine interface (HMI) and creating the route, and
initiating operation of the controller via the human-machine interface (HMI) to commence operation of the treatment device the operate the wheeled carriage to track robotically along the route by comparing the position of the wheeled carriage as detected by the light detection and ranging surveying device with a desired position along the route and by controlling the position, direction of movement and speed of the wheels of the carriage.

19. A method as claimed in claim 16, comprising the additional step of calculating a total treatment time for the treatment procedure and transmitting same to a portable timer adapted to provide an alarm when the treatment procedure has been carried out by the treatment device.

20. A method as claimed in claim 19, wherein total treatment time is calculated by the controller and/or the human-machine interface (HMI) and transmitted to the timer by Wi-Fi on commencement of the treatment procedure by the treatment device.

21. A method as claimed in claim 19, wherein a projected time for the treatment procedure is calculated using a known path length of the recorded route and a trace speed, which is the speed of movement of the wheeled carriage when travelling robotically during operation of the treatment device and which is assumed to be constant, any required dwell times at the start and end of the treatment being added to the projected time in order to obtain the total treatment time.

22. A method as claimed in any of claim 16, wherein prior to operation of the treatment device the apparatus is plugged into a mains electrical supply to power operation of the treatment device.

23. A method as claimed in claim 22, wherein during operation of the treatment device the electrical mains supply charges a rechargeable main battery that powers operation of the wheeled carriage.

24. A method as claimed in any of claim 16, wherein the human-machine interface (HMI) docks on the wheeled carriage and the method comprises the step of undocking the human-machine interface (HMI) from the wheeled carriage and standing it outside the enclosed space prior to using it to initiate operation of the controller.

25. A method as claimed in claim 24, wherein the rechargeable main battery charges a first rechargeable daughter battery that powers the human-machine interface (HMI) when the human-machine interface (HMI) is docked on the wheeled carriage and the treatment device is not operating

26. A method as claimed in claim 24, wherein the timer is powered by a second rechargeable battery that is charged at the same time as the daughter battery in the human-machine interface (HMI) when the timer is plugged into the human-machine interface (HMI) and the human-machine interface (HMI) is docked on the wheeled carriage.

27. A method as claimed in any of claim 16, wherein the treatment is disinfection either by means of a plurality of UV-C emitting lamps that are mounted in fixed positions on the wheeled carriage or by means of a hydrogen peroxide vapour (HPV) fogging device.

28. A method as claimed in claim 27, wherein the treatment is disinfection by means of a plurality of UV-C emitting lamps and a plurality of first UV sensors are provided that are mounted in fixed positions on the wheeled carriage and linked to the controller, each first sensor being monitoring operation of one of the UV-C emitting lamps and relaying information relating thereto to the controller.

29. A method as claimed in claim 27, wherein the treatment is disinfection by means of a plurality of UV-C emitting lamps and a plurality of autonomous, second UV sensors are provided that are detachably mounted on the wheeled carriage, the method comprising the step of detaching the second UV sensors from the wheeled carriage and placing them at locations in the enclosed space remote from the UV-C lamps, the second UV sensors operating to sense the doses of UV-C radiation received at each of said remote locations whereby operation of the disinfecting device may be validated.

30. A method as claimed in claim 29, wherein the second UV sensors are powered by rechargeable batteries that are charged by the main battery when the second UV sensors are mounted on and plugged into sockets provided for them in the wheeled carriage.

31. A non-tangible machine readable media configured to perform the method comprising:
locating a wheeled carriage and treatment device within the enclosed space;
recording a route around the enclosed space from a start position to an end position;
commencing operation of the treatment device and operating the wheels of the carriage so that the wheeled carriage robotically tracks along the route around the enclosed space;
receiving information from a LIDAR unit confirming that the wheeled carriage is tracking along the route around the enclosed space as intended.

32. An apparatus comprising a robotic mobile apparatus, comprising a treatment device mounted on a wheeled carriage with omni-directional, controllable wheels that are adapted to be motor driven;
the apparatus further comprising a LIDAR (Light Detection and Ranging) unit configured to be aware of the position of the wheeled carriage;
the apparatus further comprising a programmable controller with a computer memory configured to control operation of the wheels of the carriage and to operate the treatment device;
the apparatus further comprising a human-machine interface (HMI) in communication with the controller and operable to start and stop a treatment procedure;
wherein the apparatus is configured to:
locate the wheeled carriage and treatment device within an enclosed space;
position the human-machine interface (HMI) outside the enclosed space;
record a route around the enclosed space in the computer memory from a start position to an end position;
initiate operation of the controller via the human-machine interface (HMI) to commence operation of the treatment device and to operate the wheels of the carriage so that the wheeled carriage robotically tracks along the route around the enclosed space, further using the LIDAR unit to confirm that the wheeled carriage is tracking along the route around the enclosed space as intended.
